SEQUENCE LISTING (3 sequences)

SEQ ID NO: 1 - protein, 206 aa, Homo sapiens
QTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYLGGPFSPNVLNWRALKYEVQGEVFTKPQLWP

SEQ ID NO: 2 - protein, 206 aa, Homo sapiens mutant
QTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTILLGGPFSPNVLNWRALKYEVQGEVFTKPQLWP

SEQ ID NO: 3 - protein, 206 aa, Homo sapiens mutant
QTDMSRKAFVFPKESDTSYVSLKAPLTKPLKAFTVCLHFYTELSSTRGYSIFSYATKRQDNEILIFWSKDIGYSFTVGGSEILFEVPEVTVAPVHICTSWESASGIVEFWVDGKPRVRKSLKKGYTVGAEASIILGQEQDSFGGNFEGSQSLVGDIGNVNMWDFVLSPDEINTIYEGGPFSPNVLNWRALKYEVQGEVFTKPQLWP

(19) United States
(12) Patent Application Publication
(10) Pub. No.: US 2013/0072445 A9
(43) Pub. Date: Mar. 21, 2013

(54) DEVELOPMENT OF C-REACTIVE PROTEIN MUTANT WITH IMPROVED THERAPEUTIC BENEFIT IN IMMUNE THROMBOCYTOPENIA AND LUPUS NEPHRITIS

(71) Applicants: Terry W. Du Clos, Albuquerque, NM (US); Carolyn Mold, Albuquerque, NM (US)

(72) Inventors: Terry W. Du Clos, Albuquerque, NM (US); Carolyn Mold, Albuquerque, NM (US)

(21) Appl. No.: 12/995,828

(22) PCT Filed: Jun. 2, 2009
(86) PCT No.: PCT/US09/03338

§ 371 Date: Mar. 7, 2011

Related U.S. Application Data
(63) Continuation-in-part of application No. 12/083,055, filed on Aug. 11, 2008, now Pat. No. 8,013,120.
(60) Provisional application No. 60/730,013, filed on Oct. 26, 2005.
(60) Provisional application No. 61/130,749, filed on Jun. 3, 2008.

(51) Int. Cl. A61K 38/17; A61P 29/00; A61P 7/06; A61P 37/06; A61P 17/00; A61P 37/00; A61P 33/06

(52) U.S. Cl. 514/21.2; 514/18.6; 514/13.5; 514/4.9

(57) ABSTRACT

The present invention relates to the use of a mutant CRP molecule in which tyrosine 175 is replaced by leucine (Y175L CRP) or the leucine 176 is replaced by glutamic acid (L176E CRP) for the treatment of various disease states and conditions associated with SLE, including lupus of the skin (discoid), systemic lupus of the joints, lungs and kidneys, hematological conditions including hemolytic anemia and low lymphocyte counts, lymphadenopathy and CNS effects, including memory loss, seizures and psychosis, among numerous others as otherwise disclosed herein. In another aspect of the invention, the reduction in the likelihood that a patient who is at risk for an outbreak of a disease state or condition associated with SLE will have an outbreak is an additional aspect of the present invention. The present invention relates to the use of mutant Y175L CRP or L176E CRP in the treatment of a number of disease states or conditions that occur secondary to systemic lupus SLE. The present invention also relates to the treatment of immune thrombocytopenic purpura. Pharmaceutical compositions are also disclosed based these mutant CRP molecules.

### RELATED APPLICATIONS/CLAIM OF PRIORITY

This application claims the benefit of priority of U.S. provisional application Ser. No. 61/130,749, filed Jun. 3, 2008, entitled “Development of a C-reactive Protein Mutant with Improved Therapeutic Benefit in Immune Thrombocytopenia and Lupus Nephritis”, which is incorporated by reference in its entirety herein.

### FIELD OF THE INVENTION

The present invention relates to the use of a mutant CRP molecule for the treatment of various disease states and conditions associated with SLE, including lupus of the skin (discoid), systemic lupus of the joints, lungs and kidneys, hematological conditions including hemolytic anemia and low lymphocyte counts, lymphadenopathy and CNS effects, including memory loss, seizures and psychosis, among numerous others as otherwise disclosed herein. In another aspect of the invention, the inhibition or reduction in the likelihood that a patient who is at risk for an outbreak of a disease state or condition associated with SLE will have an outbreak is an additional aspect of the present invention. The present invention relates to the use of mutant Y175L CRP and/or L176E CRP in the treatment of a number of disease states or conditions that occur secondary to systemic lupus SLE. The present invention also relates to the treatment of immune thrombocytopenic purpura. Pharmaceutical compositions are also disclosed based these mutant CRP molecules.

### BACKGROUND INFORMATION

C-reactive protein (CRP) is an acute phase protein that is found at dramatically increased levels in serum following injury, infection or inflammation (reviewed in (1)). The biological activities of CRP are mediated by ligand binding and interaction with the Fc receptors for IgG (FcγR) or activation of the complement system. These biological activities include recognizing and promoting the clearance of damaged cells, nuclear antigens and microbial pathogens.

CRP also has anti-inflammatory activity, which has been the focus of our recent work and patent. We have demonstrated that a single injection of purified CRP is protective in two mouse models of SLE (2, 3), in nephrotoxic nephritis (NTN) (3), and in a model of immune thrombocytopenia (ITP) (5). The common feature of these disease models is immune complex activation of inflammatory cells through FcγR. We find that in these diseases, the initial event in CRP therapy is the induction of a suppressive macrophage. This occurs following CRP binding and signaling through one its receptors, FcγRI. In the ITP experiments, transfer of CRP-induced suppressive macrophages is sufficient to protect recipient mice from the disease (5). In the mouse as in man in addition to FcγRI, CRP binds to FcγRII, but this interaction is not essential in either the NTN or ITP model. There is evidence from other laboratories that CRP may contribute to atherosclerosis and cardiovascular disease. Most of the atherogenic effects of CRP are attributed to interactions with FcγRII on endothelial cells. There is also experimental evidence that CRP increases myocardial reperfusion injury by activating complement at the ischemic site. Thus the potential cardiovascular side effects of CRP result from complement activation and FcγRII binding, whereas its anti-inflammatory activity is mediated through FcγRI. Therefore modification of CRP to increase its binding to FcγRI and decrease its interactions with complement and FcγRII is expected to increase its anti-inflammatory activity and reduce its pro-inflammatory activity.

### BRIEF DESCRIPTION OF THE FIGURES

FIG. 1 shows the amino acid sequence of CRP, Y175L CRP (a mutant CRP) and the amino acid sequence of L176E CRP (another mutant CRP).

FIGS. 2A-C show increased cytokine responses of human monocytes incubated with mutant CRP. Human peripheral blood monocytes were purified by positive selection on anti-CD14 microbeads. Cells were incubated for 24 h with human CRP or recombinant Y175L mutant CRP. Supernatants were collected and analyzed for cytokines by ELISA (mean±SEM, n=3)

FIG. 3A-B shows increased binding of mutant CRP to FcγRI on mouse macrophages. Peritoneal exudate cells were isolated and incubated with purified human or mutant CRP. CRP binding to macrophages was detected by two-color flow cytometry. A. Macrophages expressing FcγRI (from FcγRIIb^{-/-} mice). B. Macrophages expressing FcγRIIb (from FcR γ-chain^{-/-} mice).

FIG. 4A-B shows the transfer of CRP-treated spleen cells decreases thrombocytopenia in ITP. Spleen cells were treated in vitro with CRP (200 μg/ml) or IVIG (18 mg/ml) for 30 min. BSA-treated cells were used as a control, equivalent to no cell transfer. One ×10⁶ washed cells were injected i.v. into recipient mice. Recipients were treated 24 h later with 2 μg of anti-CD41. Platelets were counted before injection (normal) and 24 h later. Results are mean±SEM, n=3, *p<0.05, **p<0.01.

FIG. 5 shows that CRP pretreatment inhibits TNF-α and increases IL-10 responses of monocytes to LPS. Human peripheral blood monocytes were cultured with CRP for 20 h and then for 4 h alone or with LPS (10 ng/ml). Culture supernatants were analyzed for cytokines by ELISA. Left. TNF-α. Right. IL-10. The means±SEM for triplicate wells are shown.

### BRIEF DESCRIPTION OF THE INVENTION

The present invention relates to the use of mutant CRP molecules in which tyrosine 175 is replaced by leucine (Y175L CRP) or leucine 176 is replaced by glutamic acid (L176E CRP) for the treatment of various disease states and conditions associated with SLE, including lupus of the skin (discoid), systemic lupus of the joints, lungs and kidneys, hematological conditions including hemolytic anemia and low lymphocyte counts, lymphadenopathy and CNS effects, including memory loss, seizures and psychosis, among numerous others as otherwise disclosed herein. In another aspect of the invention, the inhibition or reduction in the likelihood that a patient who is at risk for an outbreak of a disease state or condition associated with SLE will have an outbreak is an additional aspect of the present invention. The present invention relates to the use of mutant Y175L CRP or L176E CRP in the treatment of a number of disease states or conditions that occur secondary to SLE. In particular aspects of the invention, any one or more of secondary conditions, disease states or manifestations of SLE including serositis, malar rash (rash over the cheeks and bridge of the nose), discoid rash (scaly, disk-shaped sores on the face, neck and chest), sores or ulcers (on the tongue, in the mouth or nose), arthritis, hemolytic anemia, lymphadenopathy, low lymphocytic count, low platelet count, the presence of antinuclear antibodies in the blood, skin lesions, CNS effects (including loss of memory, seizures, strokes and psychosis), lung symptoms/effects including inflammation (pleuritis), chronic pneumonitis, chronic diffuse interstitial lung disease and scarring of the lungs, hair loss, Raynaud's syndrome, lupus nephritis and sensitivity to light, fatigue, fever, nausea, vomiting, diarrhea, swollen glands, lack of appetite, sensitivity to cold (Raynaud's phenomenon) and weight loss is treated using compounds and pharmaceutical compositions according to the present invention. The present invention also relates to the treatment of immune thrombocytopenic purpura. Because of the selective binding characteristics of Y175L CRP or L176E CRP (especially Y175L CRP), therapeutic approaches using these mutant CRP molecules provide high efficacy in treating one or more of the above disease states and conditions, with relatively low incidence of side effects (toxicity and undesirable inflammation) which occur when CRP is used.

The method of the present invention comprises administering to a patient suffering from SLE an effective amount of Y175L CRP (a mutant CRP) or L176E CRP (mutant CRP) alone or in combination with a natural or synthetic carrier such as human serum albumin, optionally in the presence of a pharmaceutically acceptable additive, carrier or excipient in an amount effective to treat SLE, and in particular, any one or more of its secondary disease states, conditions or symptoms of said patient as otherwise described herein. In optional embodiments of the present invention, CRP, or one of the other compounds disclosed herein, is administered to patients suffering from SLE including where the SLE produces or expresses itself in a kidney associated disease or condition, including lupus nephritis. The present invention also relates to the treatment of immune thrombocytopenic purpura. Pharmaceutical compositions comprising an effective amount of Y175L CRP or L176E CRP alone or in combination with a pharmaceutically acceptable additive, carrier or excipient are additional aspects of the present invention.

In alternative embodiments of the invention, a compound according to the present invention (Y175L CRP or L176E CRP) alone or in combination with an active carrier may be coadministered with an effective amount of at least one additional agent which is traditionally used in the treatment of SLE. These agents include, for example, non-steroidal anti-inflammatory drugs (NSAIDs) including traditional NSAIDs, COX-2 inhibitors and salicylates (such as aspirin), anti-malarials such as hydroxychloroquine, quinacrine, corticosteroids such as prednisone (Deltasone), betamethasone (Celestone), methylprednisolone acetate (Medrol, Depo-Medrol), hydrocortisone Cortef, Hydrocortone) and dexamethasone (Decadron, Hexadrol), among others and immunosuppressants such as methotrexate (Rheumatrex), cyclophosphamide (Cytoxan), Azathioprine (Immuran) and mycophenolate mofetil (MMF, also CellCept).

### DETAILED DESCRIPTION OF THE INVENTION

The following terms shall be used to describe the present invention.

The term “patient” refers to an animal, preferably a mammal, even more preferably a human, in need of treatment or therapy to which compounds according to the present invention are administered in order to treat a condition or disease state associated with SLE treatable using compounds according to the present invention.

The term “compound” is used herein to refer to any specific chemical compound disclosed herein. Within its use in context, the term generally refers to a single compound, generally a polypeptide of varying length.

The term “systemic lupus erythematosus”, “SLE” or “lupus” is used to describe a chronic potentially debilitating or fatal autoimmune disease in which the immune system attacks the body's cells and tissue, resulting in inflammation and tissue damage. LSE refers to several forms of an immunologic disease that affects the joints, skin, muscles, face and mouth, kidneys, central nervous system and other parts of the body. SLE is a chronic and inflammatory disease that can potentially be fatal. SLE can either be classified as an autoimmune or a rheumatic disease. Changes in symptoms are called flares and remissions. Flares are periods when SLE becomes more active with increased symptoms, and remissions are periods when few or no symptoms of lupus are present. In the United States alone, an estimated 270,000 to 1.5 million or more people have SLE, with an estimated 5 million worldwide, having the disease. It is more common than cystic fibrosis or cerebral palsy.

The specific cause of SLE is unknown. It is considered to be a multifactorial condition with both genetic and environmental factors involved. In a multifactorial condition, a combination of genes from both parents, in addition to unknown environmental factors, produce the trait, condition, or disease. It is known that a group of genes on chromosome 6 that code for the human leukocyte antigens play a major role in a person's susceptibility or resistance to the disease. The specific HLA antigens associated with SLE are DR2 and DR3. When the immune system does not function properly, it loses its ability to distinguish between its own body cells and foreign cells. Antinuclear antibodies are autoantibodies (antibodies that fight the body's own cells) that are produced in people with SLE. They often appear in the blood of a patient with SLE.

Studies suggest that some people may inherit the tendency to get SLE, and new research suggests that new cases of SLE appear to be more common in families in which one member already has the disease. However, there is no evidence that supports that SLE is directly passed from parent to child. Females in their childbearing years (18-45) are eight to ten times more likely to acquire SLE than men, and children and the elderly can also acquire the disease.

SLE is unpredictable, and no two people have exactly the same manifestations of the disease. There are 11 criteria that help doctors tell the difference between people who have SLE and people who have other connective tissue diseases. If a person displays 4 or more of the following 11 criteria, the person fulfills the requirement for the diagnosis of SLE.

1. Malar rash—a butterfly shaped rash over the cheeks and across the bridge of the nose;

2. Discoid rash—scaly, disk-shaped sores on the face, neck, and chest;

3. Serositis—inflammation of the lining around the heart, lungs, abdomen, causing pain and shortness of breath;

4. Photosensitivity—skin rash as an unusual reaction to sunlight;

5. Sores or ulcers on the tongue, mouth, or in the nose;

6. Arthritis;

7. Kidney disorder—persistent protein or cellular cysts in the urine;

8. Central nervous system problems including seizures and psychosis;

9. Blood problems such as low white blood cell count, low lymphocyte count, low platelet count, or hemolytic anemia;

10. Immune system problems (immune dysfunction/dysregulation)—presence of abnormal autoantibodies to double stranded DNA, Sm antigen or phospholipid in the blood; and

11. Presence of abnormal antinuclear antibodies in the blood.

Other symptoms/manifestations of SLE include inflammatory lung problems, lymphadenopathy, fever, nausea, vomiting, diarrhea, swollen glands, lack of appetite, sensitivity to cold (Raynaud's phenomenon), weight loss, and hair loss.

Notwithstanding the numerous disease states, conditions and/or manifestations associated with SLE, it is difficult to diagnose because there is no single set of signs and symptoms to determine if a person has the disease. There is no single test that can diagnose SLE. Some tests used to diagnose SLE include urinalysis to detect kidney problems, tests to measure the amount of complement proteins in the blood, complete blood cell counts to detect hematological disorders, and an ANA test to detect antinuclear antibodies in the blood. Additionally, X-rays may be ordered to check for lung and heart problems.

The term “effective” shall mean, within context, an amount of a compound, composition or component and for a duration of time (which may vary greatly depending upon the disease state, condition or manifestation to be treated or to have a reduced likelihood of occurring), which produces an intended effect. In instances where more than one compound is administered (coadministration) or a component is used, that compound or component is used in an effective amount to produce a desired or intended effect, in many instances, a favorable therapeutic outcome.

The term “treatment” or “treating” is used to describe an approach for obtaining beneficial or desired results including and preferably clinical results. For purposes of this invention, beneficial or desired clinical results include, but are not limited to, one or more of the following: alleviation of one or more symptoms, diminishment or inhibition of the extent of disease, stabilized (i.e., not worsening) state of disease, inhibiting, preventing or reducing the likelihood of the spread of disease, inhibiting or reducing the likelihood of occurrence or recurrence of disease, decreasing, delaying, inhibiting or reducing the likelihood of the occurrence of “flares,” amelioration of the disease state, producing a remission (whether partial or total), reduction of incidence of disease and/or symptoms, stabilizing (i.e., not worsening) of immune or renal function or improvement of immune or renal function. “Flares” refer to an increase in activity, generally inflammatory activity in a particular tissue. The “treatment” of SLE may be administered when no symptoms of SLE are present, and such treatment (as the definition of “treatment” indicates) reduces the incidence or likelihood of flares. Also encompassed by “treatment” is a reduction of pathological consequences of any aspect of SLE or any associated disease states or conditions, including skin rashes (malar and discoid), arthritis, serositis (inflammation of the lining around the heart, lungs, abdomen), sores (mouth, nose and tongue), immune dysfunction/dysregulation, central nervous system problems (including psychosis, seizures and strokes), blood problems (including low white blood cell count, low platelet count, or anemia), the presence of antinuclear antibodies in the blood and kidney disease/dysfunction (especially SLE-related nephritis).

In the case of ITP, the compounds according to the present invention may be administered in an effective amount to treat or inhibit ITP, especially including reducing or inhibiting the symptoms of bleeding, red dots on the skin, red dots on the mouth membranes, purplish mouth membrane areas, bleeding nose, bleeding gum, digestive bleeding, urinary bleeding and brain bleeding. The reduction of one or more of these symptoms is a measure of success in treating ITP. In addition, in the case of ITP, there is an increased platelet count pursuant to successful therapy.

“SLE flares” are used herein to refer to flares (i.e. acute clinical events) which occur in patients with SLE. The SLE flares may be in various major organs, including but not limited to, kidney, brain, lung, heart, liver, connective tissues and skin. Flares can include activity in all tissues that may be affected by SLE. Remission is a term used to refer to periods of little or no lupus symptoms.

“Reducing incidence” of renal flares in an individual with SLE means any of reducing severity (which can include reducing need for and/or amount of (e.g., exposure to) other drugs generally used for this conditions, including, for example, high dose corticosteroid and/or cyclophosphamide), duration, and/or frequency (including, for example, delaying or increasing time to renal flare as compared to not receiving treatment) of renal flare(s) in an individual. As is understood by those skilled in the art, individuals may vary in terms of their response to treatment, and, as such, for example, a “method of reducing incidence of renal flares in an individual” reflects administering the conjugate(s) described herein based on a reasonable expectation that such administration may likely cause such a reduction in incidence in that particular individual.

The term “immune thrombocytopenic purpura” or “ITP” is used throughout the specification to describe an autoimmune disease characterized by platelet clearance mediated by pathogenic platelet-specific antibodies. The disease is characterized by reduced blood platelets, which cause visible skin blemishes from bleeding or bruising. Symptoms can include the following: bleeding, red dots on the skin, red dots on the mouth membranes, purplish mouth membrane areas, bleeding nose, bleeding gum, digestive bleeding, urinary bleeding and brain bleeding. Immune thrombocytopenic purpura (ITP) is a clinical syndrome in which a decreased number of circulating platelets (thrombocytopenia) manifests as a bleeding tendency, easy bruising (purpura), or extravasation of blood from capillaries into skin and mucous membranes (petechiae).

In persons with ITP, platelets are coated with autoantibodies to platelet membrane antigens, resulting in splenic sequestration and phagocytosis by mononuclear macrophages. The resulting shortened life span of platelets in the circulation, together with incomplete compensation by increased platelet production by bone marrow megakaryocytes, results in a decreased platelet count.

To establish a diagnosis of ITP, other causes of thrombocytopenia are excluded, such as leukemia, myelophthisic marrow infiltration, myelodysplasia, aplastic anemia, or adverse drug reactions. Pseudothrombocytopenia due to platelet clumping is also a diagnostic consideration. No single laboratory result or clinical finding establishes a diagnosis of ITP; it is a diagnosis of exclusion.

Pathophysiology: An abnormal autoantibody, usually immunoglobulin G (IgG) with specificity for 1 or more platelet membrane glycoproteins (GPs), binds to circulating platelet . membranes. Autoantibody-coated platelets induce Fc receptor-mediated phagocytosis by macrophages, primarily but not exclusively in the spleen. The spleen is the key organ in the pathophysiology of ITP not only because platelet autoantibodies are formed in the white pulp but also because macrophages in the red pulp destroy immunoglobulin-coated platelets.

If bone marrow megakaryocytes cannot increase production and maintain a normal number of circulating platelets, thrombocytopenia and purpura develop. Impaired thrombopoiesis is attributed to failure of a compensatory increase in thrombopoietin and megakaryocyte apoptosis.

In the U.S., the annual incidence of chronic ITP is estimated to be 5.8-6.6 cases per 100,000 persons, but these data are not from large population-based studies. Most cases of acute ITP, particularly in children, are mild and self-limited and may not receive medical attention. Therefore, estimated incidences of acute ITP are difficult to determine and likely to understate the full extent of the disease.

The primary cause of long-term morbidity and mortality is hemorrhage. The most frequent cause of death in association with ITP is spontaneous or accidental trauma-induced intracranial bleeding in patients whose platelet counts are less than 10×10⁹/L (<10×10³/mL). This situation occurs in less than 1% of patients.

To maintain a platelet count in a safe range in patients with chronic treatment-resistant ITP, a long-term course of corticosteroids, other immunosuppressive medications, or splenectomy may be required. In patients with this disease, morbidity and mortality can be related to treatment, reflecting the complications of therapy with corticosteroids or splenectomy.

In children, the prevalence is the same among boys and girls. In adults, women are affected approximately 3 times more frequently than men. Children may be affected at any age, but the prevalence peaks in children aged 3-5 years. Adults may be affected at any age, but most cases are diagnosed in women aged 30-40 years. Onset in a patient older than 60 years is uncommon, and a search for other causes of thrombocytopenia is warranted. The most likely causes in these persons are myelodysplastic syndromes, acute leukemia, and marrow infiltration (myelophthisis).

The term “C-reactive protein” or “CRP” is used herein to describe a 206 amino acid protein, which is a member of the class of acute phase reactants as its levels rise dramatically during inflammatory processes occurring in the body. It is thought to assist in removal of damaged cells and affect the humoral response to disease. It is also believed to play an important role in innate immunity, as an early defense system against infections. CRP is used mainly as a marker of inflammation and for treatment of SLE and related disease states and/or conditions. CRP is the prototypic acute phase reactant in humans and is a component of the innate immune system. CRP binds to nuclear antigens that are the target of the autoantibodies of patients with SLE as well as to damaged membranes and microbial antigens. CRP activates the classical complement pathway and interacts with phagocytic cells through FcγR. CRP is protective against various inflammatory states including endotoxin shock and inflammatory alveolitis. CRP protection against endotoxin shock requires FcγR and is associated with FcγR -dependent induction of interleukin-10 (IL-10) synthesis by macrophages.

CRP is an acute phase serum protein that provides innate immune recognition, opsonization, and regulation of autoimmunity and inflammation. CRP may bind several autoantigens in SLE, for example SmD1 and 70K proteins of Sm and RNP, histones, and chromatin. CRP may activate complement and may bind to FcγRI and FcγRII in man and mouse. CRP is a natural product found in the serum of people, and it is believed to be nontoxic.

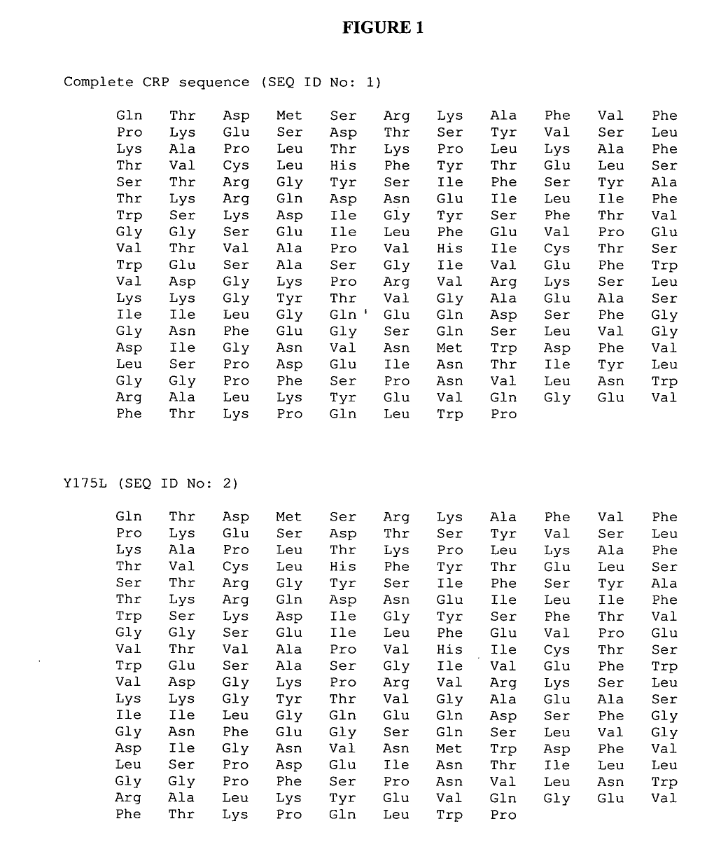

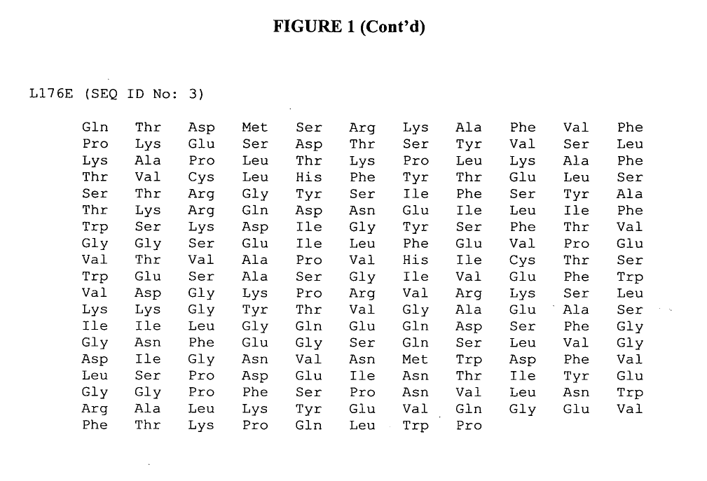

CRP has 206 amino acid units. The entire sequence of C-reactive protein appears in FIG. 1 (SEQ ID NO:1). The polypeptide sequence of CRP also has the following Accession numbers: BC125135, NM_{—}000567, BC070257, BC020766, M11880, M11725, X56214 and X56692, all of which sequences are incorporated by reference herein. SEQ ID NO:1 is also represented as follows:

(SEQ ID NO: 1)
QTDMSRKAFVFPKESDTSYVSLKAPLTKPLKAFTVCLHFYTELSSTRG
YSIFSYATKRQDNEILIFWSKDIGYSFTVGGSEILFEVPEVTVAPVHI
CTSWESASGIVEFWVDGKPRVRKSLKKGYTVGAEASIILGQEQDSFGG
NFEGSQSLVGDIGNVNMWDFVLSPDEINTIYLGGPFSPNVLNWRALKY
EVQGEVFTKPQLWP

Y175L Mutant CRP contains 206 amino acids as above wherein tyrosine 175 is replaced by a leucine. The entire sequence appears below.

(SEQ ID NO: 2)
QTDMSRKAFVFPKESDTSYVSLKAPLTKPLKAFTVCLHFYTELSSTRG
YSIFSYATKRQDNEILIFWSKDIGYSFTVGGSEILFEVPEVTVAPVHI
CTSWESASGIVEFWVDGKPRVRKSLKKGYTVGAEASIILGQEQDSFGG
NFEGSQSLVGDIGNVNMWDFVLSPDEINTILLGGPFSPNVLNWRALKY
EVQGEVFTKPQLWP

L176E Mutant CRP contains 206 amino acids for CRP as above wherein leucine 176 is replaced by a glutamic acid. The entire sequence appears below.

(SEQ ID NO: 3)
QTDMSRKAFVFPKESDTSYVSLKAPLTKPLKAFTVCLHFYTELSSTRG
YSIFSYATKRQDNEILIFWSKDIGYSFTVGGSEILFEVPEVTVAPVHI
CTSWESASGIVEFWVDGKPRVRKSLKKGYTVGAEASIILGQEQDSFGG
WNFEGSQSLVGDIGNVNMDFVLSPDEINTIYEGGPFSPNVLNWRALKY
EVQGEVFTKPQLWP

In one aspect of the invention, Y175L or L176E mutant C-reactive protein is prepared as a dosage formulation for delivery to a human patient and administered in order to treat systemic lupus erythematosus (SLE) or any one or more of the secondary disease states, conditions or symptoms which occur in a patient with SLE.

The Y175L or L176E mutant C-reactive protein polypeptide of the present invention may be administered directly as a pharmaceutical composition when combined with a pharmaceutically acceptable additive, carrier or excipient or alternatively, may be used in combination with a carrier (adsorbed or covalently bound to the carrier as otherwise described herein). These are useful in the treatment of SLE and its secondary disease states, conditions and manifestations, especially including lupus nephritis and ITP and as otherwise described herein.

The term “carrier” or “active carrier” shall be used in context to describe a complex molecule, including a polymer which can be used in combination with Y175L or L176E mutant C-reactive protein polypeptides of the present invention. A carrier may be an oligomeric polypeptide, such as oligo- or polylysine, oligo- or polyarginine, or a mixture thereof (generally from about 5-1000 mer or greater, but also ranging from about 10 to about 100 mer), polyglutamic acid, polyaspartic acid, polyhistidine, polyasparagine, polyglutamine, etc. or a dendrimer as otherwise disclosed in US patent publication 2003/0232968 to Chun Li, et al., which is incorporated by reference in its entirety herein. Additional dendrimers are available from Sigma-Aldrich, USA or Dendritic Nano Technologies, Inc., Mount Please, Mich., USA. Dendrimers may include PAMAM dendrimers, phosphorous dendrimers, polypropylenimine dendrimers, lysine dendrimers, among numerous others. Also called a cascade molecule, a dendrimer is a polymer that has many branches that move out from a core, generally a carbon core. Many of these dendrimers are available commercially from Sigma-Aldrich or from Dendritic Nano Technologies.

Other ways of attaching the protein or polypeptide include modification of a particle surface by adsorption or covalent attachment of suitable linking group(s) to which the protein may be subsequently attached. Examples of additional carriers include polyethylene glycol (with an average molecular weight ranging from about 100 to about 2000), polyethylene glycol co-polypropylene glycol copolymer (random or block copolymers) of similar molecular weight as the polyethylene glycol, albumin (preferably human serum albumin for human therapies), collagen (preferably human recombinant collagen), gelatin, dextran (including cyclodextrin), alginate, polylactide/glycolide, polyhydroxy-butyrate, polyvinyl alcohol, polyanhydride microspheres and liposomes, among others. One of ordinary skill will readily recognize how to complex or attach the present therapeutic polypeptides to carriers using techniques and methodologies which are well known in the art.

The term “coadministration” or “combination therapy” is used to describe a therapy in which at least two active compounds in effective amounts are used to treat SLE a related disease state, condition or symptom at the same time. Although the term coadministration preferably includes the administration of two active compounds to the patient at the same time, it is not necessary that the compounds be administered to the patient at the same time, although effective amounts of the individual compounds will be present in the patient at the same time.

According to various embodiments, the Y175L or L176E mutant C-reactive protein polypeptide compounds according to the present invention may be used for treatment or prevention/inhibition purposes in the form of a pharmaceutical composition. This pharmaceutical composition comprises a mutant polypeptide as disclosed above which is optionally combined with an active carrier, especially, a polypeptide carrier as otherwise described herein. Active metabolites of CRP mutants as otherwise disclose may also be used. For example, an embodiment of the pharmaceutical composition may comprise a mixture of a Y175L and/or L176E mutant CRP and a metabolite of CRP. The oral dosage form may be in a form chosen from a solid, semi-solid, and liquid.

The pharmaceutical composition may also comprise a pharmaceutically acceptable excipient, additive or inert carrier (distinguishable from active carriers which are complexed with an active polypeptide herein). The pharmaceutically acceptable excipient, additive or inert carrier may be in a form chosen from a solid, semi-solid, and liquid. The pharmaceutically acceptable excipient or additive may be chosen from a starch, crystalline cellulose, sodium starch glycolate, polyvinylpyrolidone, polyvinylpolypyrolidone, magnesium stearate, sodium lauryl sulfate, sucrose, gelatin, silicic acid, polyethylene glycol, water, alcohol, propylene glycol, vegetable oil, corn oil, peanut oil, olive oil, surfactants, lubricants, disintegrating agents, preservative agents, flavoring agents, pigments, and other conventional additives. The pharmaceutical composition may be formulated by admixing the active with a pharmaceutically acceptable excipient or additive. If a polypeptide carrier is used, it is preferred to combine the polypeptide with the polypeptide carrier before combining with other components in preparing a pharmaceutical dosage form.

The pharmaceutical composition may be in a form chosen from sterile isotonic aqueous solutions, pills, drops, pastes, cream, spray (including aerosols), capsules, tablets, sugar coating tablets, granules, suppositories, liquid, lotion, suspension, emulsion, ointment, gel, and the like. Administration route may be chosen from subcutaneous, intravenous, intestinal, parenteral, oral, pulmonary (especially for treatment of lung conditions), buccal, nasal, intramuscular, transcutaneous, transdermal, intranasal, intraperitoneal, and topical (especially for certain skin rashes and skin conditions).

The subject or patient may be chosen from, for example, a human, a mammal such as domesticated animal, or other animal. The subject may have one or more of the disease states, conditions or symptoms associated with SLE or ITP, as otherwise described herein.

The compounds according to the present invention may be administered in an effective amount to treat or reduce the likelihood of SLE, any one or more of the disease states conditions or conditions associated with SLE including, for example serositis, malar rash (rash over the cheeks and bridge of the nose), discoid rash (scaly, disk-shaped sores on the face, neck and chest), sores or ulcers (on the tongue, in the mouth or nose), arthritis, hemolytic anemia, low lymphocytic count, low platelet count, the presence of antinuclear bodies in the blood, skin lesions, CNS effects (including loss of memory, seizures, strokes and psychosis), lung symptoms/effects including inflammation (pleuritis), chronic pneumonitis, chronic diffuse interstitial lung disease and scarring of the lungs, hair loss, Raynaud's syndrome, lupus nephritis and sensitivity to light, fatigue, fever, nausea, vomiting, diarrhea, swollen glands, lack of appetite, sensitivity to cold (Raynaud's phenomenon) and weight loss. In the case of ITP, the compounds according to the present invention in pharmaceutical dosage form may be administered in an amount to treat or inhibit ITP, especially including reducing or inhibiting the symptoms of bleeding, red dots on the skin, red dots on the mouth membranes, purplish mouth membrane areas, bleeding nose, bleeding gum, digestive bleeding, urinary bleeding and brain bleeding. Each of these is a measure of success in treating ITP. In addition, in the case of ITP, there is an increased platelet count pursuant to successful therapy.

One of ordinary skill in the art would be readily able to determine an effective amount of one or more compounds according to the present invention within the context of therapy and/or prevention/reducing the likelihood or inhibition by taking into consideration several variables including, but not limited to, the animal subject, age, sex, weight, site of the disease state or condition in the patient, previous medical history, other medications, etc.

For example, the dose of a compound for a human patient is that which is an effective amount and may range from as little as 50-100 μg to at least about 500 mg to 1 gram or more, which may be administered in a manner consistent with the delivery of the drug and the disease state or condition to be treated. In the case of oral administration, active is generally administered from one to four times or more daily. Transdermal patches or other topical administration my administer drugs continuously, one or more times a day or less frequently than daily, depending upon the absorptivity of the active and delivery to the patient's skin. Of course, in certain instances where parenteral administration represents a favorable treatment option, intramuscular administration or slow IV drip may be used to administer active. The amount of CRP which is administered daily to a human patient preferably ranges from about 0.05 mg/kg to about 10 mg/kg or more, about 0.1 mg/kg to about 7.5 mg/kg, about 0.25 mg/kg to about 6 mg/kg, about 1.25 to about 5.7 mg/kg.

The dose of a compound according to the present invention may be administered prior to the onset of SLE, during SLE flares or during remission prior to an expected flare. For example, the dose may be administered for the purpose of treating and/or reducing the likelihood of any one or more of these disease states or conditions occurs or manifests, including serositis, malar rash (rash over the cheeks and bridge of the nose), discoid rash (scaly, disk-shaped sores on the face, neck and chest), sores or ulcers (on the tongue, in the mouth or nose), arthritis, hemolytic anemia, low lymphocytic count, low platelet count, the presence of antinuclear bodies in the blood, skin lesions, CNS effects (including loss of memory, seizures, strokes and psychosis), lung effects including chronic pneumonitis and scarring of the lung, hair loss, Raynaud's syndrome, lupus nephritis, sensitivity to light, fatigue, fever, nausea, vomiting, diarrhea, swollen glands, lack of appetite, sensitivity to cold (Raynaud's phenomenon), weight loss, and hair loss. The dose may be administered prior to diagnosis, but in anticipation of SLE or anticipation of flares. The dose also is preferably administered during flares to reduce the severity of same. In the case of ITP, compounds are administered when ITP is first diagnosed, or at the first signs of ITP symptomatology, including the symptoms of bleeding, red dots on the skin, red dots on the mouth membranes, purplish mouth membrane areas, bleeding nose, bleeding gum, digestive bleeding, urinary bleeding and brain bleeding and reductions of one or more of these symptoms are measures of success. In addition, therapy may include administration of compounds according to the present invention at the first sign of decreased platelet count. In the case of ITP using the present compounds, there is an increased platelet count pursuant to successful therapy.

In alternative embodiments of the invention, a Y175L and/or L176E mutant CRP compound according to the present invention (alone or in combination with an active carrier as otherwise described herein) in pharmaceutical dosage form may be coadministered with an effective amount of at least one additional agent which is traditionally used in the treatment of system lupus erythematosus or immune thrombocytopenic purpura (ITP). These agents may include, for example, non-steroidal anti-inflammatory drugs (NSAIDs) including traditional NSAIDs, including COX-2 inhibitors and salicylates (such as aspirin, tolmetin, aspirin, diclofenac, etodolac, ibuprofen, indomethacin, ketoprofen, ketorolac, nabumetone, naproxen, oxaprozin, piroxicam, celecoxib, sulindac), anti-malarials, such as hydroxychloroquine, quinacrine, corticosteroids such as prednisone (Deltasone), betamethasone (Celestone), methylprednisolone acetate (Medrol, Depo-Medrol), hydrocortisone (Cortef, Hydrocortone) and dexamethasone (Decadron, Hexadrol), among others and immunosuppressants such as methotrexate (Rheumatrex), cyclophosphamide (Cytoxan), Azathioprine (Imuran) and mycophenolate mofetil (MIVIF, also CellCept). In the case of ITP, the treatment may include a corticosteroid (as described above) or an immunosuppressant. In one embodiment, preferred agents to be used for ITP treatment include dexamethasone or prednisone.

The present invention also relates to a method of suppressing autoantibody production in a patient comprising administering to said patient an effective amount of Y175L and/or L176E mutant CRP compound in combination with a pharmaceutically acceptable additive, excipient, or carrier, optionally in combination with an active carrier.

The crystal structure for CRP interaction with FcγR has been solved (6). The contact residues between CRP and FcγR have been identified and a small number of mutant CRP molecules have been tested for binding to human FcγR using surface plasmon resonance (SPR). One of these mutants in which tyrosine 175 is replaced by leucine (Y175L CRP) has decreased binding to FcγRII and FcγRIII, but retains binding to FcγRI (Table 1). Y175L CRP has also lost the ability to activate complement (7).

TABLE 1
Analysis of CRP binding to human
FcγR by surface plasmon resonance.

| | Target | | |
|---|---|---|---|
| Analyte | FcγRI | FcγRIIa | FcγRIII |
| CRP (Kd μM) | 3.2 ± 0.2 | 1.9 ± 0.6 | 4.1 ± 0.4 |
| Y175L CRP (Kd μM) | 3.5 ± 0.9 | >16.8 | >11.5 |

The dissociation constants (in μM) for CRP and Y175L CRP binding to immobilized human FcγR are shown (6).

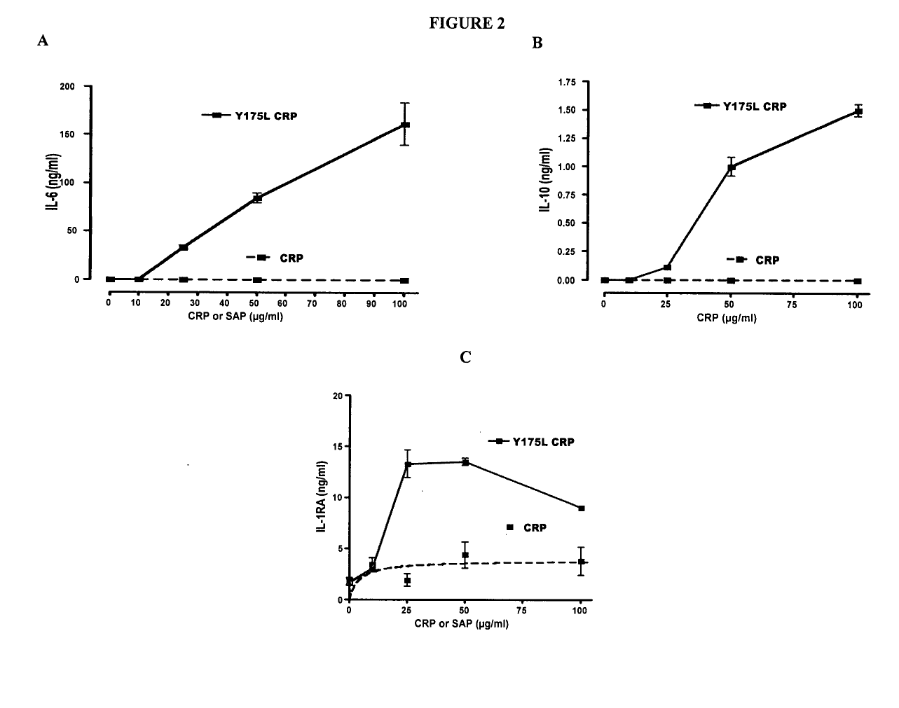

The inventors also tested Y175L CRP for induction of cytokine synthesis by human monocytes (FIG. 2). Peripheral blood monocytes released several cytokines after incubation for 24 h with the mutant protein. The cytokine response to purified human CRP was much lower. The cytokines included the anti-inflammatory cytokines, IL-10 and IL-IRA, but also other cytokines associated with stimulation of FcγRI (IL-6, IL-8, IL-lb, TNF-a). Polymixin B (10 μg/m1) was added to the cultures to prevent any contribution of contaminating endotoxin. One explanation for the increased activity of the mutant protein would be that it is unable to bind to the inhibitory receptor, FcγRIIb (2). Direct binding assays for Y175L CRP and FcgRIIb have not been done, but the sequences of FcγRIIa and FcγRIIb in the extracellular domains are nearly identical.

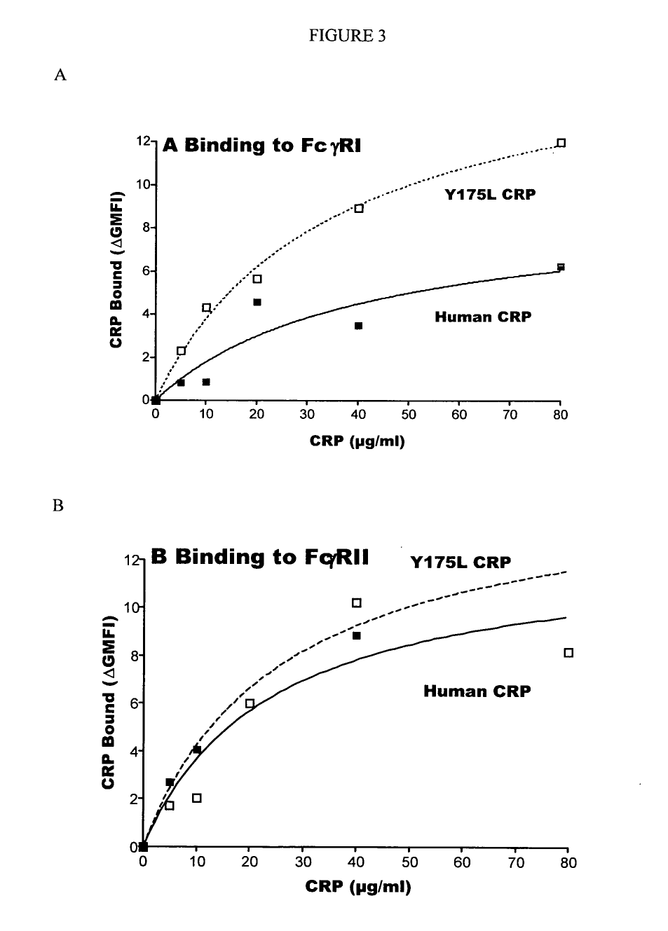

Since Y175L CRP is a candidate for selective anti-inflammatory activity, we tested its binding to mouse macrophages. The results show that Y175L has increased binding to FcγRI on mouse macrophages and normal binding to FcγRIIb (FIG. 3). Thus analysis of Y175L CRP shows an increased interaction with FcγRI relative to FcγRII in both human and mouse. We predict that Y175L CRP will be more effective than native CRP in suppressing autoimmune and inflammatory disease. Establishment of this by in vivo studies will support the approach of screening for useful mutants using SPR binding assays with purified receptors.

### Experimental Approach

Objective 1. Studies predict that a mutant CRP with the characteristics of Y175L CRP will have greater effectiveness in the autoimmune and immune complex disease models and decreased potential for adverse cardiovascular effects. Development of a therapeutically useful CRP mutant would increase commercial interest in CRP as a therapeutic agent. Preparation of sufficient quantities of highly purified, low endotoxin Y175L CRP occurs to test in the mouse ITP model. This model was chosen because it is initiated by CRP binding to FcγRI on macrophages and provides rapid results. It is predicted that the mutant will be more effective than wild type CRP in this model.

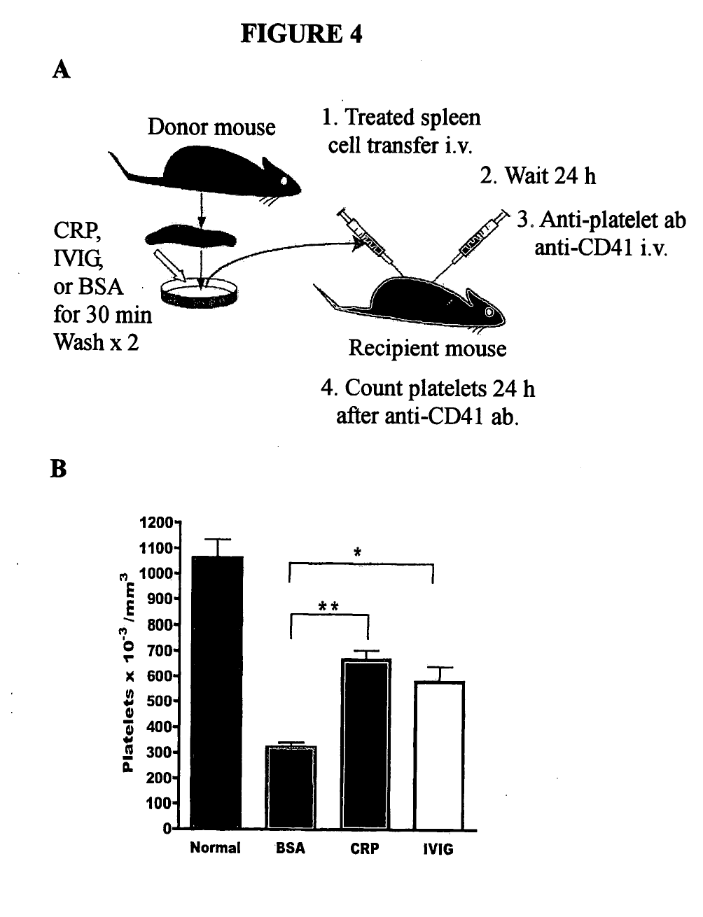

The experimental design is based on previous results. Mouse spleen cells or interferon (IFN)-γ-treated bone marrow macrophages (BMM) will be treated with increasing concentrations (50-400 μg/ml) of CRP or Y175L CRP in vitro, washed and injected into naïve recipients. After 24 h, thrombocytopenia will be induced in the recipients by injection of a rat mAb (anti-CD41) to mouse platelets. Platelets in the blood are counted 24 h after injection of the anti-platelet antibody. In the absence of macrophage transfer or using macrophages treated with a control protein (BSA), thrombocytopenia is observed with the lowest number of platelets at 24 h. This thrombocytopenia can be prevented by the transfer of CRP-treated or intravenous immunoglobulin (IVIg)-treated macrophages (FIG. 4). Direct injection of CRP intravenously 1 h prior to injection of anti-platelet antibody also protects mice from experimental ITP. Neither the passive transfer nor the direct injection provides complete restoration of platelet numbers even with higher doses of CRP. It is believed that Y175L CRP will be more effective at a lower concentration than unmodified CRP. It is possible that the residual platelet clearance will be treatable using the Y175L CRP.

Additional studies will be needed to assess the benefit of the mutation in cardiovascular disease. We plan to focus on cardiovascular disease that is seen in the context of lupus, since lupus nephritis is the proposed therapeutic application of CRP. Patients as well as mice with SLE have increased atherosclerotic disease and coronary artery vasculitis. We also plan to study the effects of short term high dose CRP therapy and long term low dose CRP exposure in the MRL/lpr SLE mouse model. These mice have increased atherogenesis and myocardial infarction when fed a high lipid diet. The Y175L CRP is to be tested in these models, but this would not be completed during the 1 year period of STC Gap funding.

Objective 2. We also study two in vitro models using human monocytes. In the first model, human monocytes show increased release of proinflammatory cytokines (TNF-α and IL-1) after incubation with CRP bound to bacteria (Streptococcus pneumoniae) compared to bacteria alone (4). This response is FcγRIIa-dependent. In the second model, monocytes exposed for 24 h to a high concentration of CRP without ligand become unresponsive to an inflammatory stimulus. It is believed that this unresponsiveness is the human counterpart to the induction of suppressive macrophages in the mouse. However, the receptors involved have not been identified. The signaling pathways used by human FcγRI and FcγRIIa receptors are overlapping, and studies using receptor-blocking antibodies are not definitive. The use of the Y175L mutant, which binds FcγRI but not FcγRIIa, together with the previously described L176E mutant, which binds FcγRIIa but not FcγRI, will allow us to define this pathway. If successful these studies will establish a human system with direct parallels to the mouse models. These findings will increase the marketability of our technology by providing evidence that the effects of CRP in mouse models of lupus will translate to treatment of human disease.

The experimental design is to isolate peripheral blood monocytes from human subjects. We then determine which of two allelic forms of FcγRIIa (His or Arg131) each individual expresses, as this affects CRP binding. Cells will be stimulated with CRP-attached to S. pneumoniae as we have described or oxidized low density lipoprotein (oxLDL), a model we will develop because of its relevance to atherogenesis. Monocyte cytokines responses will be measured after 24 h. CRP attached to S. pneumoniae increases release of the pro-inflammatory cytokines, TNF-α and IL-1b. We believe that Y175L CRP, which binds poorly to FcgRIIa, will not induce these cytokines, because it does not bind to FcγRIIa and that the L176E CRP, which binds poorly to FcγRI, will be equivalent to wild type CRP or have a greater effect.

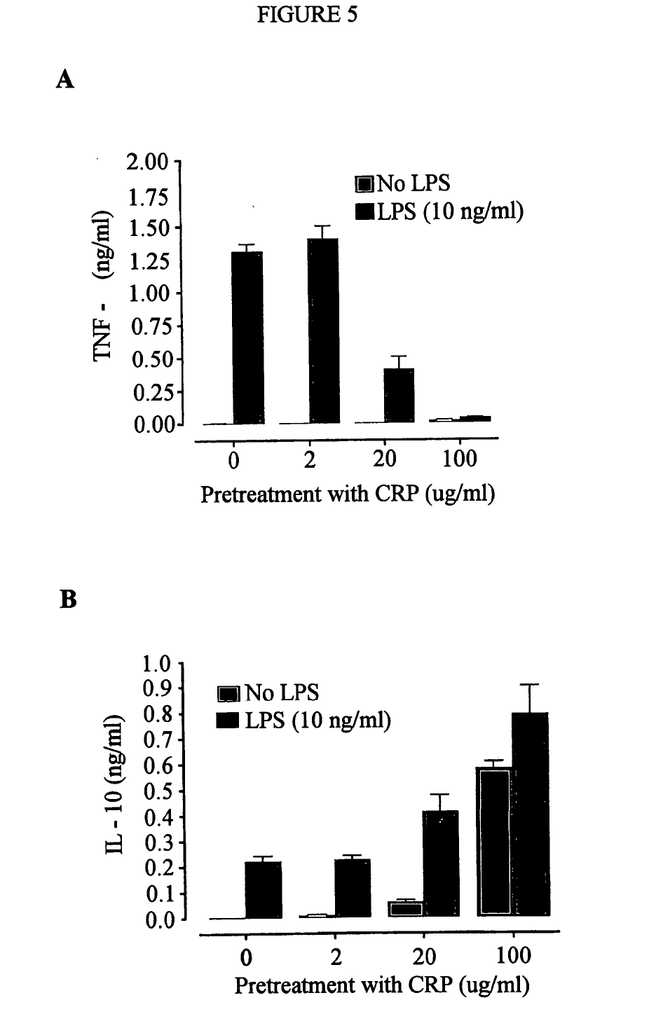

In the second set of experiments, peripheral blood monocytes are incubated with different concentrations of CRP, Y175L CRP or L176E CRP for 20 h. After this preincubation, the culture will be stimulated with lipopolysaccharide (LPS, a standard inflammatory stimulus) or immune complexes. We have found that preincubation of CRP at concentrations of 20-200 μg/ml reduces the TNF-α response to LPS stimulation, but increases the anti-inflammatory IL-10 response (FIG. 5). We compare the mutant CRP molecules in this assay. We believe that the Y175L CRP will have equal or greater ability to induce unresponsiveness compared to native CRP and that the L176E CRP will have reduced activity.

### SUMMARY

The present invention addresses the need for new biologic agents to treat autoimmune disorders e.g. SLE and immune thrombocytopenic purpura (ITP). ITP is a relatively common disorder, which may be either acute or chronic in nature. In childhood cases and some adult cases thrombocytopenia follows a viral infection and is self limiting after the viral syndrome resolves. Chronic ITP is responsible for most of the cases and is seen in women primarily. ITP may be the precursor to the development of SLE, a more serious systemic disorder. ITP is also a complication of AIDS and is difficult to treat because immune suppression is contraindicated. Traditional treatment of ITP, like SLE, often employs corticosteroid therapy, which has numerous severe side-effects including osteoporosis, cataract formation, exacerbation or development of diabetes, and numerous other problems. Clearly, like SLE, newer more effective biological approaches would be useful. In both SLE and ITP, Rituximab, a monoclonal antibody, has shown promise, but this treatment depletes the immune system of antibody forming cells for up to one year. IVIG treatment is a biological therapy with substantial effectiveness in SLE and ITP, but it is expensive and only effective transiently. Newer approaches to these related diseases would provide more directed effective therapy with less systemic side-effects.

These experiments assist in rapidly evaluating a promising CRP mutant that is expected to have improved efficacy and may have decreased side effects. The studies described herein also provide proof of principle that a preliminary identification of useful mutants can be done by affinity of binding to different FcgR using SPR. The additional studies in human monocytes further support the transferability of the mouse findings to humans.

All patents and publications referenced or mentioned herein are indicative of the levels of skill of those skilled in the art to which the invention pertains, and each such referenced patent or publication is hereby incorporated by reference to the same extent as if it had been incorporated by reference in its entirety individually or set forth herein in its entirety.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

### REFERENCES

1. Marnell, L., Mold, C., and Du Clos, T. W. 2005. C-reactive protein: ligands, receptors and role in inflammation. Clin Immunol 117:104-111.
2. Rodriguez, W., Mold, C., Kataranovski, M., Hutt, J., Marnell, L. L., and Du Clos, T. W. 2005. Reversal of ongoing proteinuria in autoimmune mice by treatment with C-reactive protein. Arthritis Rheum 52:642-650.
3. Rodriguez, W., Mold, C., Marnell, L. L., Hutt, J., Silverman, G. J., Tran, D., and Du Clos, T. W. 2006. Prevention and reversal of nephritis in MRL/lpr mice with a single injection of C-reactive protein. Arthritis Rheum 54:325-335.
4. Mold, C., and Du Clos, T. W. 2006. C-reactive protein increases cytokine responses to

Streptococcus pneumoniae through interactions with Fc gamma receptors. J Immunol 176:7598-7604.
5. Marjon, K. D., L. L. Marnell, C. Mold, and T. W. Du Clos. 2009. Macrophages activated by C-reactive protein through FcγRI transfer suppression of immune thrombocytopenia. Journal of Immunology 182:1397-1403.
5. Bang, R., L. L. Marnell, C. Mold, M. P. Stein, K. Du Clos, C. Chivington-Buck, and T. W. Du Clos. 2005. Overlap of Human FcγRI, FcγRIIa, and C1q binding sites in C-reactive protein as determined by site-directed mutagenesis. Journal of Biological Chemistry 280:25095-25102.
6. Lu, J., L. L. Marnell, K. D. Marjon, C. Mold, T. W. Du Clos, and P. D. Sun. 2008. Structural recognition and functional activation of FcγR by innate pentraxins. Nature, 456:989-992.

1. A method of treating, inhibiting or reducing the likelihood of systemic lupus erythematosus (SLE) or a secondary disease state, condition or manifestation associated with SLE or immune thrombocytopenic purpura in a patient comprising administering to said patient an effective amount of at least one compound selected from the group consisting of Y175L CRP and L176E CRP, in combination with a carrier, additive or excipient and optionally in combination with a natural or synthetic active carrier.

2. The method according to claim 1 wherein said secondary disease state, condition or manifestation is selected from the group consisting of serositis, malar rash, discoid rash, sores or ulcers on the tongue, in the mouth or nose, arthritis, hemolytic anemia, lymphadenopathy, low lymphocytic count, low platelet count, the presence of antinuclear antibodies in the blood, skin lesions, CNS effects, lung effects, hair loss, Raynaud's syndrome, lupus nephritis and sensitivity to light, fatigue, fever, nausea, vomiting, diarrhea, swollen glands, lack of appetite and weight loss.

3. The method according to claim 1 wherein said secondary disease state, condition or manifestation is serositis.

4. The method according to claim 1 wherein said secondary disease state, condition or manifestation is lupus nephritis.

5. The method according to claim 1 wherein said secondary disease state, condition or manifestation is other than lupus nephritis.

6. The method according to claim 1 wherein said secondary disease state, condition or manifestation is arthritis.

7. The method according to claim 2 wherein said CNS effect is a memory loss of psychosis.

8. The method according to claim 1 wherein said disease state, condition or manifestation is malar rash or discoid rash.

9. The method according to claim 1 wherein said disease state, condition or manifestation is lymphadenopathy.

10. The method according to claim 1 which is used to treat immune thrombocytopenia purpura.

11. The method according to claim 10 wherein said treatment reduces at least one or more of bleeding, red dots on the skin, red dots on the mouth membranes, purplish mouth membrane areas, bleeding nose, bleeding gum, digestive bleeding, urinary bleeding and brain bleeding in said patient.

12. A method according to claim 10 wherein said treatment increases circulating platelets.

13. The method according to any of claims 1-12 wherein said compound is Y175L CRP.

14. The method according to claim 1 wherein said compound is L176E CRP.

15. The method according to claim 1 wherein said compound is a combination of L175L CRP and L176E CRP.

16. (canceled)

17. A pharmaceutical composition comprising an effective amount of CRP mutant polypeptide selected from the group consisting of Y175L CRP, L176E CRP or mixtures thereof, in combination with a pharmaceutically acceptable carrier, additive or excipient and optionally in combination with an active carrier.

18. The composition according to claim 17 wherein said CRP mutant polypeptide is Y175L CRP.

19. The composition according to claim 17 wherein said CRP mutant polypeptide is L176E CRP.

20. The composition according to claim 17 wherein said CRP mutant polypeptide is a mixture of Y175L CRP and L176E CRP.

21. The composition according to any of claims 17-20 wherein said composition is formulated in combination with an active carrier.

22. The composition according to claim 17, 18 or 20 wherein said composition further comprises at least one agent selected from the group consisting of non-steroidal anti-inflammatory drugs (NSAIDs), anti-malarials, corticosteroids, immunosuppressants and mixtures thereof.

23. The composition according to claim 22 wherein said NSAID is selected from the group consisting of aspirin, tolmetin, aspirin, diclofenac, etodolac, ibuprofen, indomethacin, ketoprofen, ketorolac, nabumetone, naproxen, oxaprozin, piroxicam, celecoxib, sulindac and mixtures thereof.

24. The composition according to claim 22 wherein said corticosteroid is selected from the group consisting of prednisone, betamethasone, methylprednisolone acetate, hydrocortisone, dexamethasone and mixtures thereof.

25. The composition according to claim 22 wherein said immunosuppressant is selected from the group consisting of methotrexate, cyclophosphamide, azathioprine, mycophenolate mofetil (CellCept) and mixtures thereof.

26-36. (canceled)
